(19) United States
(12) Patent Application Publication
(10) Pub. No.: US 2016/0287901 A1
(43) Pub. Date: Oct. 6, 2016

(54) SURGICAL CAVITY SIZERS FOR BRACHYTHERAPY AND INTRAOPERATIVE RADIATION THERAPY

(71) Applicant: DUMAINE PRECISION INC., Carignan (CA)

(72) Inventors: Frederic Dumaine, Carignan (CA); Jan Seuntjens, Westmount (CA); Monica Serban, Westmount (CA); Marija Popovic, Montreal (CA)

(21) Appl. No.: 15/035,051

(22) PCT Filed: Nov. 7, 2014
(86) PCT No.: PCT/CA2014/051075

§ 371 Date: May 6, 2016

Related U.S. Application Data
(60) Provisional application No. 61/901,667, filed on Nov. 8, 2013.

(51) Int. Cl. A61N 5/10; A61B 50/33; A61B 17/34

(52) U.S. Cl. CPC A61N 5/1015; A61B 17/3468; A61B 50/33; A61B 17/3403; A61N 2005/1003

(57) ABSTRACT

Surgical cavity sizers for selecting a radiation applicator for brachytherapy and intraoperative radiation therapy. The surgical cavity sizer has a head portion and a handle portion monolithic to the head portion. The surgical cavity sizer is composed of biocompatible and sterilizable material. A method for selecting a radiation applicator as well as a container for a surgical cavity sizer and/or a radiation applicator are also disclosed.

### CROSS-REFERENCE TO RELATED APPLICATIONS

This application claims the benefit of U.S. provisional patent application 61/901,667 filed Nov. 8, 2013, the entire contents of which are hereby incorporated by reference.

### TECHNOLOGICAL FIELD

This invention relates to radiotherapies, and more particularly, to surgical cavity sizers for determining the appropriate radiation applicators to be used during brachytherapy or intraoperative radiation therapy, as well as containers for individually packaging radiation applicators.

### BACKGROUND

Brachytherapy is a form of radiotherapy where a radiation source is placed internally either inside or next to the area requiring treatment. Intraoperative Radiation Therapy (IORT) is an in situ ionizing radiation treatment of the surgical cavity in which a radiation treatment is provided to an area adjacent to a tumor which has been excised (e.g., a surgical cavity or a tumor bed). IORT can be delivered using kV X-rays emitted by a miniature, probe-type X-ray unit, placed inside an applicator (e.g., a radiation applicator). After the tumor has been excised from the patient, a radiation applicator is introduced in the vicinity of the excised area and is associated with a radiation therapy system to provide radiation therapy. In order to achieve acceptable therapeutic results, it is important that the radiation therapy be applied in close contact with the tumor bed. The radiation application must therefore closely match the volume and area of the surgical cavity.

Prior to delivery of radiation, the size of the natural or surgical cavity thus needs to be determined so that the radiation delivery is optimally performed. This is currently done by trial and error using the same sterilized radiation applicators as those used during the radiation delivery. With this techniques, the radiation applicators which are not selected to deliver radiation must go through a significant number of sterilization cycles, which is needlessly large. Furthermore, the maximum number of allowed sterilizations can be quickly attained, after which new applicators need to be acquired.

Another technique consists of taking approximate measurements of the natural or surgical cavity using a length measurement device (e.g., a ruler), which is then used to select the final radiation applicator for the radiation treatment. This technique relies on the experience of the individual surgeon, and may introduce inconsistency in the selection of the most appropriate radiation applicator.

### BRIEF SUMMARY

In accordance with an aspect, there is provided a device for intraoperative radiation therapy and brachytherapy, comprising: a surgical cavity sizer for a radiation applicator having a distal end to be located in a surgical cavity and allowing radiation emission therethrough, said surgical cavity sizer including a head portion having a shape and a size identical to the distal end of the radiation applicator, and a handle portion projecting from the head portion and permitting manipulation of the surgical cavity sizer, the handle portion being integrally formed with the head portion to form a monolithic structure composed of a biocompatible and sterilizable material.

According to another aspect, there is provided a container for a device for intraoperative radiation therapy and brachytherapy, the device including a surgical cavity sizer or a radiation applicator, said container comprising a pivoting engagement mechanism retaining the device, the pivoting engagement mechanism operable to pivot the device between a storage position, wherein a longitudinal axis of the device disposed fully within the container, and a releasable position, wherein the device is in an angled, partially upright, position and the distal end is at least partially out of the container.

According to yet another aspect, there is provided a method of selecting a radiation applicator for performing brachytherapy or intraoperative radiation therapy in a natural or surgical cavity, the method comprising: providing a plurality of radiation applicators, each having a distal end having a different size and/or shape; providing a plurality of surgical cavity sizers each having a head portion of different size and/or shape matching the size and/or shape of the distal end of respective ones of the radiation applicators; determining, from the surgical cavity sizers, the surgical cavity sizer conforming to the size and/or shape of the natural or surgical cavity, thereby providing a selected surgical cavity sizer; and selecting the radiation applicator having the distal end with the same size and/or shape as the head portion of the selected surgical cavity sizer to perform brachytherapy or intraoperative radiation therapy.

According to yet another aspect, there is provided a kit for selecting an appropriately sized radiation applicator for providing brachytherapy or intraoperative radiation therapy, said kit comprising a plurality of the devices as defined above, wherein the head portion of each of the surgical cavity sizers has a different size and/or shape.

According to yet another aspect, there is provided a kit for selecting an appropriately sized radiation applicator amongst a plurality of radiation applicators to provide brachytherapy or intraoperative radiation therapy, said kit comprising a plurality of the devices as defined above, wherein the head portion of each of the surgical cavity sizers is identical in shape and/or size to a corresponding distal end of one of the radiation applicators.

### BRIEF DESCRIPTION OF THE DRAWINGS

Reference is now made to the accompanying figures in which:

FIG. 1A is a schematic side elevation view of a radiation applicator used for a radiotherapy.

FIG. 1B is a side elevation view of a surgical cavity sizer, according to an embodiment of the present disclosure.

FIG. 2 is a perspective view of a kit of surgical cavity sizers of different sizes, including a container for sterilizing and storing the surgical cavity sizers, according to another embodiment of the present disclosure.

FIG. 3A is a perspective view of a sterilizing and storage container for a radiation applicator, according to yet another embodiment of the present disclosure.

FIG. 3B is a perspective view of the container of FIG. 3A, showing the radiation applicator in a releasable position.

FIG. 4 is a flow diagram of a method for selecting a radiation applicator, according to yet another embodiment of the present disclosure.

### DETAILED DESCRIPTION

As noted above, in the past, the radiation applicators themselves (i.e. the applicator end, used to deliver the radiation therapy) have been used to size the surgical cavity/tumor bed. To reduce the number of times the radiation applicators are contaminated without providing radiation therapy and are thus subjected to non-useful and potentially detrimental sterilization, separate surgical cavity sizers (i.e. distinct from the radiation applicators) are disclosed herein.

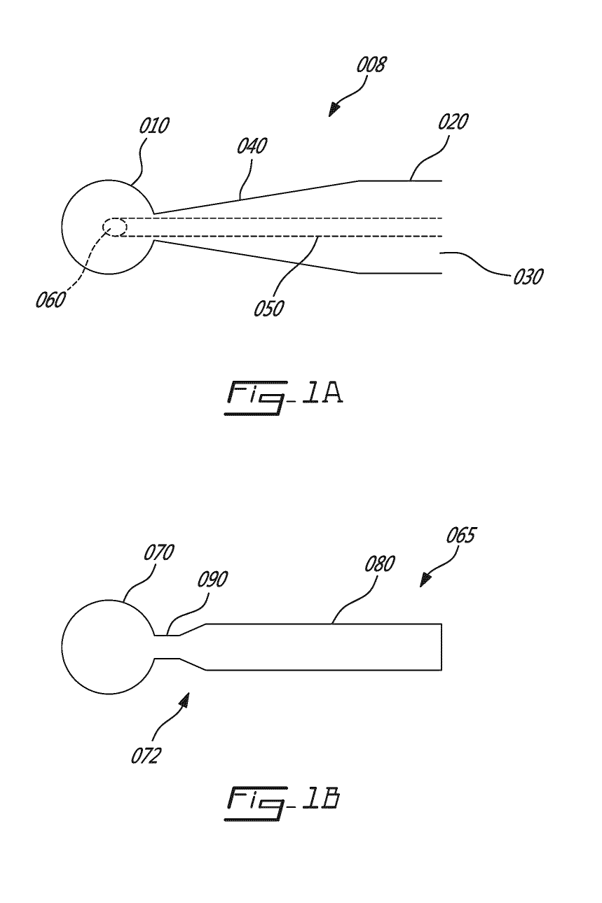

FIG. 1A shows a radiation applicator 008 used to provide Intraoperative Radiation Therapy (IORT). The radiation applicator 008 has a distal end 010 intended to be located and fit within the surgical cavity. The distal end 010 of the radiation applicator 008 is configured to allow the dissemination of ionizing radiation to the surrounding tissue. The distal end 010 is typically a hollow structure which can accommodate a tip 060 of a source of ionizing radiation directed through a central tube 050. In FIG. 1A, the distal end 010 has a spherical shape, however, it is possible to provide a distal end 010 having a different shape.

At least one section of the peripheral surface of the first distal end 010 can be composed of a material which allows the transmission of ionizing radiation to the surrounding tissue. The distal end 010 is connected to the shaft 020 of the radiation applicator 008, which may be hollow and fit over the central tube 050. The shaft 020 may have a tapered section 040 extending between the proximal end 030 (i.e. the end 030 manipulated by the user) of the shaft 020 and the distal end 010. The shaft 020 of the radiation applicator 008 is configured for receiving and, in some instances, holding in place a source of ionizing radiation having the tube 050 and the tip 060. The tube 050 is disposed in operable connection with a brachytherapy or IORT system. As such, the shaft 020 of the radiation applicator 008 is at least partially hollow to allow the insertion and removal of the tube 050 and the tip 060 of the radiation emitter of the brachytherapy/IORT system. Although FIG. 1A illustrates a radiation applicator 008 having a rigid distal end 010 and a rigid shaft 020, the radiation applicator 008 may alternately have a flexible (and in some embodiments inflatable, such as balloon applicator) distal end 010 and/or a flexible shaft 020.

FIG. 1B shows a device including a surgical cavity sizer 065. The device can include components other than the surgical cavity sizer 065, and in some instances, can be a radiation applicator. However, for the purposes of simplicity, only the surgical cavity sizer 065 is discussed herein, and it will therefore be appreciated that the device and the surgical cavity sizer 065 can be referred to interchangeably. The surgical cavity sizer 065 shown is similar in size and/or shape to the radiation applicator shown in FIG. 1A. The surgical cavity sizer 065 is considered to correspond or match the radiation applicator because a head portion 070 of the surgical cavity sizer 065 is of the same size and/or shape as the distal end of the radiation applicator. The surgical cavity sizer 065 has a monolithic handle 080 and may include, in one embodiment, a connector portion 090 or neck located between the head portion 070 and the handle 080. The connector portion 090 connects or links the head portion 070 and the handle 080, and can therefore assume shapes and configurations different from the one shown in the figures.

The surgical cavity sizer 065 has a head portion 070 and a handle portion 080 which are monolithic (i.e. they are integrally formed of the same material) in that they form a monolithic structure 072. A plurality of such surgical cavity sizers 065 can be provided, as will be described in further detail below, and the head portion 070 of each of the surgical cavity sizers 065 matches the size and/or shape of the distal end of a corresponding radiation applicator susceptible to be used to provide brachytherapy/IORT. The surgical cavity sizers 065 are thus used to determine if the corresponding radiation applicator will fit in the natural or surgical cavity, and/or to select the most appropriate size and/or shape of the radiation applicator.

The monolithic structure 072 formed by the head portion 070 and the handle portion 080 is composed of a single material, which is different from the material of the radiation applicator 008. More particularly, the monolithic structure 072 is composed of a biocompatible material (e.g., a material considered safe for human use). For example, the surgical cavity sizer 065 can be composed of a high-performance medical grade plastic. In one embodiment, this material selected for monolithic structure 072 may include an amorphous thermoplastic such as polyphenylsulfone (PPSU). When compared to the material of the radiation applicator 008, the material of the surgical cavity sizer 065 may be capable of sustaining additional rounds of sterilization (such as, for example hot steam, ethylene oxide, plasma and gamma rays) without substantially being deformed or degraded.

In some embodiments, the surface of the monolithic structure 072 is smooth to facilitate the cleaning of the surgical cavity sizer 065 as well as its insertion in the surgical cavity. Additionally, while the head 070 of the surgical cavity sizer 065 is depicted as being substantially spherical in shape, it is to be understood that the head 070 may alternately be formed having a different shape in order to better suit the surgical cavity within which the head 070 is to be inserted or in order to be consistent with a differently shaped radiation applicator used in the radiation delivery. When spherical in shape, the head 070 of the surgical cavity sizer 065 may have a diameter of between about 1.5 cm and about 5.0 cm. When a plurality of such surgical cavity sizers 065 are provided, as a kit for example, the spherical heads 070 of each of the surgical cavity sizers 065 may have diameters of about 1.5 cm, about 2.0 cm, about 2.5 cm, about 3.0 cm, about 3.5 cm, about 4.0 cm, about 4.5 cm and about 5.0 cm. Other sizes and/or combination of sizes are of course also possible, as may be required depending on the size and type of surgical cavity.

The handle 080 of the surgical cavity sizer 065 is intended to facilitate handling of the instrument and may be of a cylindrical shape, as shown. Regardless, the handle 080 is sized and shaped such that it can be manipulated by the surgeon or user of the surgical cavity sizer 065, as may be required in order to insert the head 070 into place within the surgical cavity. In some embodiments, the handle portion 080 of the surgical cavity sizer 065 has a shape and/or size which is substantially similar to the shaft 020 of the radiation applicator. However, it is contemplated that other types of handle portions 080 can also be used.

Depending on the intended use and the type of surgical cavity being treated, the surgical cavity sizer 065 can be a rigid monolithic structure 072 or a flexible monolithic structure 072. In the latter instance, it is to be understood that when a flexible surgical cavity sizer 065 is used, its structure 072 does not allow the deformation of the size and/or shape of the head portion 070, but, for example, can allow for bending the handle portion 080 and/or the connector portion 090. In another embodiment, the head 070 may be formed separately from the handle 080 (i.e. they are not integrally formed or monolithic), provided that the entire surgical cavity sizer 065 remains able to be similarly sterilized as required.

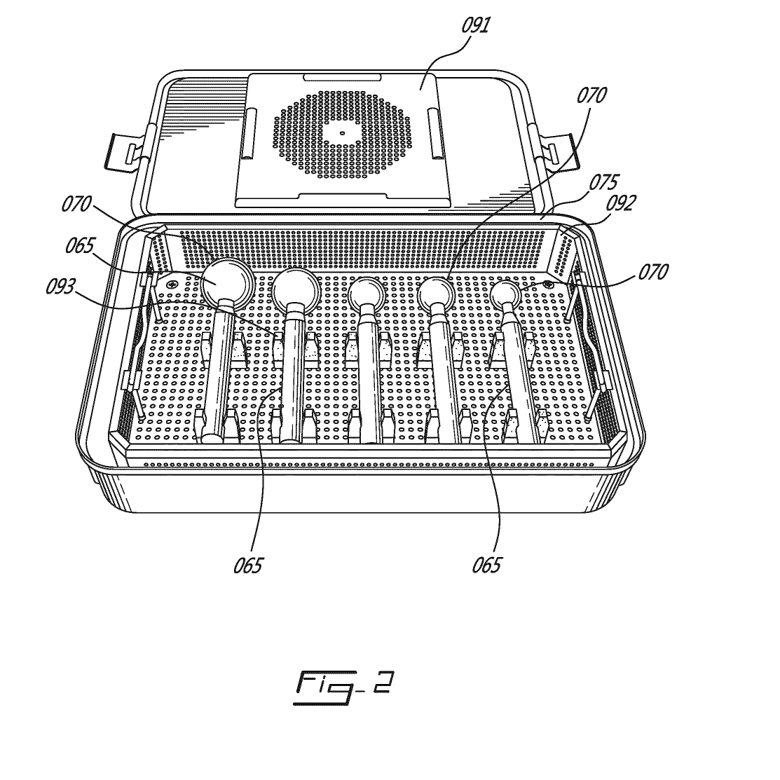

Referring to FIG. 2, due to the fact that the surgical cavity sizers 065 are to be inserted into a natural or surgical cavity, they are provided in a sterile container 075. The surgical cavity sizers 065 can be individually packaged and sterilized, such that each container 075 contains only one surgical cavity sizer 0065. Alternatively and more commonly, a plurality of surgical cavity sizers 065 can be provided in a single container 075. The container 075 is a sealable outer casing having a removable lid 091. A removable inner tray 092 can fit within the outer casing of the container 075. The inner tray 92 may be perforated and holds the sterilized surgical cavity sizers 065 in place. The inner tray 92 can be removed from the container 075 so as to be separately sterilized, if desired.

Each of the surgical cavity sizers 065 may be releasably attached to the internal bottom surface of the tray 092 by a pair of retention devices 093, which engage the surgical cavity sizers 065 at opposite ends of the handle portion 080 in order to secure the surgical cavity sizers 065 in place. The retention devices 093 may include, for example, clamps, latches, U-shaped resilient holders, and the like. One or more of these retention devices 093 may be provided for each of the surgical cavity sizers 065, or, alternately, a single retention device 093 may retain all of the plurality of sizers 065 in spaced-apart position within the tray 092. In the embodiment of FIG. 2, the retention devices 093 do not provide a pivot and are intended to prevent contact between the plurality of surgical cavity sizers 065 or movement of the surgical cavity sizers 065 during the handling/sterilizing of the container 075 or the tray 092.

A kit is disclosed herein which comprises one or more surgical cavity sizers 065, and which may also have one or more corresponding matching radiation applicators within separate containers. In this embodiment, it is understood that the surgical cavity sizer(s) 065 be provided in a sterile container 075 which is distinct from the sterile container of the corresponding matching radiation applicator(s). It is also understood that, in the kit, for each radiation applicator susceptible of being used to provide brachytherapy/IORT, corresponding surgical cavity sizers 065 having the same size and/or shaped head portions 070 as the distal ends of the radiation applicators are provided.

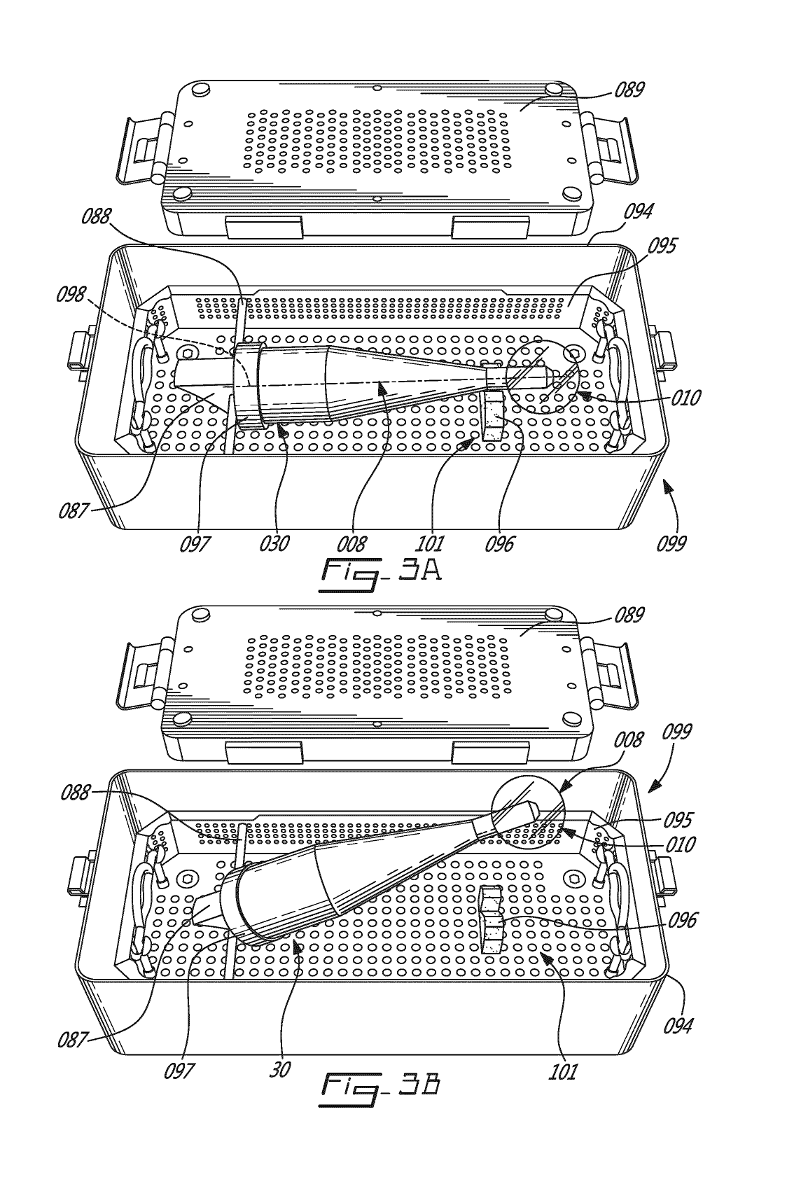

FIGS. 3A and 3B show a sterile container 099 for radiation applicators 008. Such a sterile container 099 helps to limit the contamination of the radiation applicators 008 which are not ultimately used to provide IORT (i.e. those having unsuitable sizes) by individually providing and preserving a single radiation applicator 008. Multiple sterile containers 099 can be provided, where each sterile container 099 houses and preserves a single radiation applicator 008 with a different size and/or shape. Each individual sterile container 099 may include markings or other identifying information indicating the size and/or shape of the radiation applicator 008, and that of a matching corresponding surgical cavity sizer, amongst other possibilities. A plurality of matching surgical cavity sizers (which can be, for example, packaged as a kit within a separate, single sterile container) are provided and used to select the appropriate radiation applicator 008. Once the appropriate surgical cavity sizer is selected, only the container 099 housing the corresponding matching radiation applicator 008 need be opened, the selected radiation applicator 008 then being used for providing brachytherapy or IORT. The sterile container 099 containing the individual radiation applicator 008 thereby advantageously allows the other sterile containers 099 containing the other unselected radiation applicators 008 to remain undisturbed and in a sterile state.

The container 099 includes an outer casing 094 and a removable lid 089 which is fastenable to the box or casing 094 in a sealed manner. A removable inner tray 095 fits within the cavity of the casing 094, to which a radiation applicator 008 is removably engaged by a pivoting engagement mechanism 101. The pivoting engagement mechanism 101 includes a first, fixed, engaging element 096 and a second, pivoting, hinged engagement element 097.

The fixed engaging element 096 can be similar to the retention devices of the sterile container for the surgical cavity sizers discussed above. More particularly, the fixed engaging element 096 may include, for example, clamps, latches, U-shaped resilient holders, and the like. One or more of these fixed engaging elements 096 may be provided for the radiation applicator 008, or, alternately, a fixed engaging element 096 may retain the radiation applicator 008 within the sterile container 099. The fixed engaging element 096 does not allow the distal end 010 of the radiation applicator 008 to pivot at the point of engagement, and is instead intended to secure the radiation applicator 008 in place within the container 099.

As shown in FIG. 3A, when the radiation applicator 008 is in its storage/sterilization/transport position within the container 099, a longitudinal axis 098 of the radiation applicator 008 is substantially parallel, if not collinear, to a longitudinal axis of the rectangular container 099. In this position, the proximal end 030 of the radiation applicator 008 is disposed in the hinged engagement element 097 and the opposite distal end 010 of the radiation applicator 008, near the head portion 010, is retained by the fixed engagement element 096.

As seen in FIG. 3B, the pivoting engagement mechanism 101 of the container 099 allows the radiation applicator 008 to be pivoted upwards, out of the casing 094 of the container 099, in order for it to be more easily removed from the casing for use. This is accomplished by releasing the fixed engagement element 096 from contact with the distal end 010, and pivoting the radiation applicator 008 with the hinged engagement element 097. This pivots the radiation applicator 008 from the storage position, where the radiation applicator 008 is substantially horizontal within the container 099, as shown in FIG. 3A, to the releasable position, where the radiation applicator 008 is in an angled, partially upright, position as shown in FIG. 3 In this fashion, the radiation applicator 008 may be more compactly stored, sterilized and transported, but still easily accessible and removed from the casing 099 for use when required by allowing the radiation applicator 008 to be pivoted upwardly partially out of the casing 094 of the container 099, as shown in FIG. 3B.

The hinged engagement element 097 allows the radiation applicator 008 to pivot about a point located on the hinged engagement element 097 so that at least the distal end 010 of the radiation applicator 008 can be moved outside the container 099, as shown in FIG. 3B. More particularly, at least the distal end 010 is placed outside the casing 094 and the cavity defined thereby such that a user or surgeon can grasp at least the distal end 010 of the radiation applicator 008. It will thus be appreciated that the hinged engagement element 097 can have many different configurations to accomplish such functionality.

In the embodiment of FIGS. 3A and 3B, the hinged engagement element 097 has a base 087 into which the proximal end 030 of the radiation applicator 008 can be placed and secured. The base 087 is mounted to a support rod 088. In such a configuration, the radiation applicator 008 can be pivoted in two possible ways. In the first, the base 087 is pivotable mounted about the support rod 088, either with suitable bearings or not, such that the base 087 pivots with respect to the support rod 088 about an axis of the support rod 088. In the second, the base 087 is fixedly mounted to the support rod 088, and the extremities of the support rod 088 are mounted to rotate within the casing 094. In such a configuration, both the base 087 and support rod 088 pivot together about the axis of the support rod 088. Other configurations for the hinged engagement element 097 are also within the scope of the present disclosure. The pivotal stiffness of the hinged engagement element 097 can also vary, so that the radiation applicator 008 has a tendency to remain in the releasable position. A locking mechanism can also be provided for such a purpose.

It will be appreciated that the pivoting engagement mechanism 101 described above can also be used in the sterile container 075 housing one or more surgical cavity sizers 065.

A method for selecting a radiation applicator for performing brachytherapy or intraoperative radiation therapy in a natural or surgical cavity is also disclosed. In typical IORT practice, the patient is first anesthetized and the cancerous tumor and surrounding tissues are excised until the wall of the excision (i.e. surgical) or natural cavity has a clear margin. In order to determine if a clear margin has been removed, the excised tissue is sent to pathology. If the surgeon decides the excised tissue exhibits a clear margin, then a radiation treatment plan is prescribed and undertaken.

The total radiation treatment is given in one session or dose. In the context of the present disclosure, at least one dose of radiation therapy is provided, optionally during the surgery, using a radiation applicator of an IORT or a brachytherapy system. Once a radiation applicator has been determined to be of the appropriate size and/or shape for providing brachytherapy/IORT, an X-ray source is inserted in the radiation applicator and the latter is inserted in the natural or surgical cavity. The X-ray source can emit at least radially uniformly, if not totally isotropically, to provide the brachytherapy/IORT. After the brachytherapy/IORT, the wound is closed and the patient is allowed to recover.

The method can be used, for example, to increase the accuracy in selecting the appropriate radiation applicator when compared to the accuracy obtained by using some conventional techniques (i.e. rulers or simply eyeballing). The method also helps to reduce the contamination (and ultimately the re-sterilization) of radiation applicators which have a size and/or a shape which does not fit appropriately in the surgical cavity.

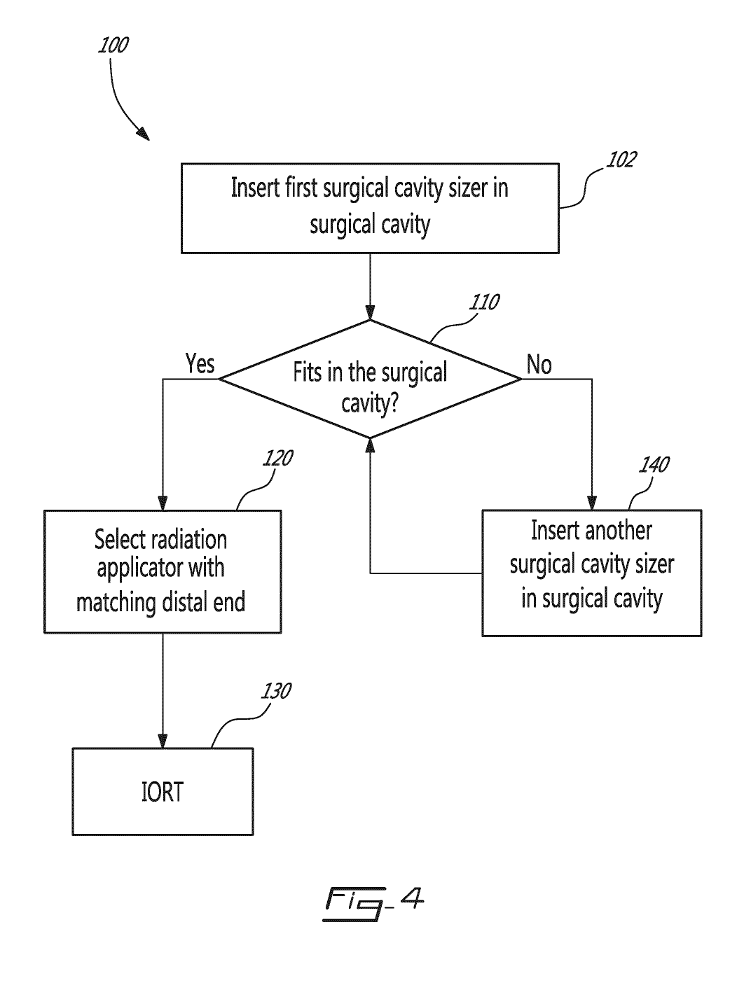

Referring to FIG. 4, the method 100 includes providing more than one radiation applicators. Each of the radiation applicators has a distal end. The distal end of each radiation application has a size and/or shape which differs from the size and/or shape of the distal ends of the other radiation applicators. Providing the radiation applicators an include having available separate sterile containers, each one containing a radiation applicator having a distal end with a unique size and/or shape.

The method 100 also includes providing more than one surgical cavity sizers. Each surgical cavity sizer has a head portion which differs from the size and/or shape of the head portions of the other surgical cavity sizers. The head portions match the size and/or shape of the distal end of at least one of the radiation applicators.

The method 100 also includes determining, from the provided surgical cavity sizers, which one fits in the surgical cavity. In order to do so, the head portion of at least one surgical cavity sizer is inserted into the natural or surgical cavity and the fit between the surgical cavity sizer and the cavity is determined. More particularly, the surgical cavity sizer conforming to the size and/or shape of the cavity determines the fit between that surgical cavity sizer and the cavity.

Providing surgical cavity sizers can be repeated with a plurality of surgical cavity sizers until the appropriate fit is obtained. This generally occurs by inserting the head portions of the surgical cavity sizers into the natural or surgical cavity before any radiation applicators are inserted in the cavity. Once it is determined which of the surgical cavity sizers fits appropriately the surgical cavity, the radiation applicator whose first distal end corresponds to size and/or shape of the head portion of the surgical cavity sizer whose fit was considered the most appropriate is selected for brachytherapy/IORT. The selected radiation applicator will therefore also provide an appropriate fit in the cavity for providing brachytherapy/IORT.

FIG. 4 provides an embodiment of the method 100 where one or optionally more different surgical cavity sizers are used. In such embodiments, the surgical cavity sizers differ at least in the shape and/or size of their head portion. Once a surgical cavity has been created or a natural cavity has been exposed, at step 102, a first surgical cavity sizer is inserted therein. Then, at decision node 110, it is determined if the inserted surgical cavity sizer fits appropriately in the cavity, i.e. if it is capable of entering the cavity and if it fills almost entirely the area (which has been optionally excised during surgery). Decision node 110 can include a visual determination of the fit between the surgical cavity sizer and the cavity, for example by examining the stretching of the surgical cavity sizer on the skin of the patient. If it is determined at decision node 110 that the surgical cavity sizer does fit within the cavity, then, at 120, the first surgical cavity sizer is removed from the surgical cavity and a radiation applicator having a first distal end which matches the size and/or shape of the head portion of the first surgical cavity sizer is selected for providing brachytherapy/IORT, shown at 130.

However, if at decision node 110, it is determined that the first surgical cavity sizer does not fit the cavity (because, for example, it is too big to enter the cavity, too small to fill the excised area or its shape does not match the excised area), at 140, the first surgical cavity sizer is removed from the cavity and another surgical cavity sizer having a different head size and/or shape is inserted in the cavity. For example, if it was determined that the first surgical cavity sizer was too small to fit in the cavity, a second surgical cavity sizer having a larger head than the first one can be inserted in the cavity. Once the subsequent surgical cavity sizer has been inserted in the cavity, it is again determined, at decision node 110, if the further surgical cavity sizer fits in the cavity, i.e. if it is capable of entering the cavity and if it fills the area which has been optionally excised during surgery. Action 140 and decision node 110 can be repeated more than once to determine if alternative surgical cavity sizers (having different head size and/or shape) fit more appropriately in the cavity.

In the method 100 described herein, a plurality of surgical cavity sizers each having a head portion of different shape and/or sizes are provided. In some embodiments, the method includes providing enough different surgical cavity sizers for matching the first distal end of the each of the plurality of the radiation applicators. When a plurality of surgical cavity sizers are provided, it is not necessary to insert all of the surgical cavity sizers in the surgical cavity to make the determination of an appropriate fit between the sizer and the surgical cavity. For example, it is possible to exclude some surgical cavity sizers before inserting them in the surgical cavity because they are clearly too big to enter the cavity, too small to fill the cavity or their shape is too dissimilar from the shape of the cavity.

Similarly, it is not always necessary to use at least two surgical cavity sizers. If it is determined that a single surgical cavity sizer fits appropriately in the surgical cavity, then it is not necessary to insert another surgical cavity sizer, to select the appropriate radiation applicator. However, in some embodiments, to confirm that a specific surgical cavity sizer is of the appropriate fit, the method can include introducing at least one more surgical cavity sizer in the cavity and comparing the fit obtained with the fit obtained by inserting the first surgical cavity sizer in the cavity. Based on this comparison, the surgical cavity sizer having the most appropriate fit is determined and the matching radiation applicator is selected for providing brachytherapy/IORT.

The method described herein can be used for selecting a radiation applicator and providing brachytherapy/IORT to various patients whose cancerous tumors have been excised. As it is known in the art, brachytherapy/IORT is commonly used as an effective treatment for cervical, prostate, breast and skin cancer and can also be used to treat tumors in many other body sites. Brachytherapy/IORT can be used alone or in combination with other therapies such as surgery, external beam radiotherapy (EBRT) and chemotherapy.

In light of the preceding, it can be appreciated that the invention disclosed herein provides alternative methods or means for selecting the type of radiation applicators for brachytherapy or IORT, as well as for reducing the unnecessary sterilization of radiation applicators. Such methods/means should preferably limit the inconsistency observed with rulers and/or limit the contamination of radiation applicators which are not being used for the delivery of radiation therapy.

While the invention has been described in connection with specific embodiments thereof, it will be understood that the scope of the claims should not be limited by the preferred embodiments set forth in the examples, but should be given the broadest interpretation consistent with the description as a whole.

1. A device for intraoperative radiation therapy and brachytherapy, comprising: a surgical cavity sizer for a radiation applicator having a distal end to be located in a surgical cavity and allowing radiation emission therethrough, said surgical cavity sizer including a head portion having a shape and a size identical to the distal end of the radiation applicator, and a handle portion projecting from the head portion and permitting manipulation of the surgical cavity sizer, the handle portion being integrally formed with the head portion to form a monolithic structure composed of a biocompatible and sterilizable material.

2. The device of claim 1, wherein the head portion has a spherical shape.

3. The device of claim 2, wherein the head portion has a diameter between about 1.5 cm to about 5.0 cm.

4. (canceled)

5. (canceled)

6. The device of claim 1, further comprising a connector portion located between the head portion and the handle portion and being monolithic to the head portion and the handle portion.

7. A container for a device for intraoperative radiation therapy and brachytherapy, the device including a surgical cavity sizer or a radiation applicator, said container comprising a pivoting engagement mechanism retaining the device, the pivoting engagement mechanism operable to pivot the device between a storage position, wherein a longitudinal axis of the device is disposed fully within the container, and a releasable position, wherein the device is in an angled, partially upright, position and the distal end is at least partially out of the container.

8. The container of claim 7, wherein the pivoting engagement mechanism includes a fixed engagement element retaining the distal end of the device, and a hinged engagement element retaining an opposed proximal end of the device.

9. The container of claim 7, wherein the longitudinal axis of the device is horizontal and substantially parallel with a longitudinal axis of the container when in the storage position.

10. The container of claim 7, further comprising a removable inner tray disposed within a cavity defined by the container.

11. The container of claim 10, wherein the device is removably engaged with the inner tray via the pivoting engagement mechanism.

12. A kit for selecting an appropriately sized radiation applicator for providing brachytherapy or intraoperative radiation therapy, said kit comprising a plurality of the devices as defined in claim 1, wherein the head portion of each of the surgical cavity sizers has a different size and/or shape.

13. A kit for selecting an appropriately sized radiation applicator amongst a plurality of radiation applicators to provide brachytherapy or intraoperative radiation therapy, said kit comprising a plurality of the devices as defined in claim 1, wherein the head portion of each of the surgical cavity sizers is identical in shape and/or size to a corresponding distal end of one of the radiation applicators.

14. (canceled)

15. The kit of claim 13, wherein the distal ends of each of the radiation applicators matches the size and/or shape of the head portion of one of the surgical cavity sizers.

16. (canceled)

17. (canceled)

18. (canceled)

19. (canceled)

20. A method of selecting a radiation applicator for performing brachytherapy or intraoperative radiation therapy in a natural or surgical cavity, the method comprising:
a) providing a plurality of radiation applicators, each having a distal end having a different size and/or shape;
b) providing a plurality of surgical cavity sizers each having a head portion of different size and/or shape matching the size and/or shape of the distal end of respective ones of the radiation applicators;
c) determining, from the surgical cavity sizers, the surgical cavity sizer conforming to the size and/or shape of the natural or surgical cavity, thereby providing a selected surgical cavity sizer; and
d) selecting the radiation applicator having the distal end with the same size and/or shape as the head portion of the selected surgical cavity sizer to perform brachytherapy or intraoperative radiation therapy.

21. The method of claim 20, wherein determining the surgical cavity sizer includes introducing at least two surgical cavity sizers having head portions of different size and/or shape in the natural or surgical cavity, and selecting one of the at least two surgical cavity sizers having the head portion conforming to the size and/or shape of the natural or surgical cavity.

22. The method of claim 20, wherein providing the plurality of radiation applicators includes providing the radiation applicators individually in a sterile container.

23. The method of claim 22, wherein providing the plurality of radiation applicators includes pivoting a second end opposite to the distal end of each radiation applicator within the sterile container.

24. The method of claim 20, wherein providing the plurality of surgical cavity sizers includes packaging the surgical cavity sizers in a second sterile container.

25. The method of claim 20, further comprising applying an ionizing radiation to the natural or surgical cavity with the selected radiation applicator.

26. The method of claim 25, wherein applying the ionizing radiation includes applying the ionizing radiation to a cancerous tumor.

27. The method of claim 20, further comprising forming the natural or surgical cavity by excising a cancerous tumor.

28. (canceled)
